(19) United States
(12) Patent Application Publication
(10) Pub. No.: US 2013/0079353 A1
(43) Pub. Date: Mar. 28, 2013

(54) FLUOROQUINOLONES FOR THE TREATMENT AND/OR PROPHYLAXIS OF INFLAMMATORY DISEASES

(71) Applicants: Karsten Gülow, Heidelberg (DE); Marcin M. Kaminski, Heidelberg (DE); H. Peter Krammer, Heidelberg (DE)

(72) Inventors: Karsten Gülow, Heidelberg (DE); Marcin M. Kaminski, Heidelberg (DE); H. Peter Krammer, Heidelberg (DE)

(21) Appl. No.: 13/518,188

(22) PCT Filed: Dec. 20, 2010
(86) PCT No.: PCT/EP10/70210

§ 371 Date: Dec. 14, 2012

(30) Foreign Application Priority Data
Dec. 22, 2009 (EP) 09180349.4

(51) Int. Cl. A61K 31/496

(52) U.S. Cl. CPC A61K 31/496
USPC 514/253.08

(57) ABSTRACT

The present invention relates to a pharmaceutical composition comprising at least one compound of formula (I)

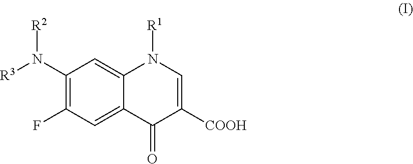

wherein
R¹ denotes e.g. hydrogen or straight chain C₁-C₆-alkyl,
R² and R³ denote e.g. hydrogen or straight chain C₁-C₆-alkyl,
   or a pharmaceutically acceptable salt thereof and at least one pharmaceutically acceptable excipient for use as a medicament for the treatment of inflammatory diseases or conditions, in particular T-cell mediated inflammatory diseases or conditions.

The present invention relates to the use of fluoroquinolones for the treatment and/or diagnosis of inflammatory diseases and disorders, in particular T-cell mediated inflammatory diseases and disorders, such as inflammatory skin diseases.

Fluoroquinolones such as the drug compounds ofloxacin, levofloxacin, norfloxacin, and ciprofloxacin are known antibacterial agents, effective in particular against both gram positive and gram negative bacteria, including pseudomonas aeruginosa. Fluoroquinolon antibiotics have been used against bacterial infections of the intestinal or urinary tract. The antibiotic effect of fluoroquinolones, such as ciprofloxacin, is based on the inhibition of bacterial topoisomerase H and an induction of DNA doublestrand breaks. The document U.S. Pat. No. 4,670,444 describes the antibacterial effect of fluoroquinolones, especially ciprofloxacin.

Serious adverse events may occur with oral treatment with fluoroquinolones, such as ciprofloxacin, e.g. neuropathy, spontaneous tendon rupture and tendonitis, acute liver failure or serious liver injury (hepatitis), but also toxic epidermeal necrolysis (TEN) and exanthema have been observed. Ciprofloxacin, as well as other members of the fluoroquinolones group of antibiotics, are characterised by immunomodulatory properties of so far unknown mechanism. The effects of ciprofloxacin on T-cell activation-induced gene expression remain vague. Numerous conflicting reports stated that ciprofloxacin either activates or inhibits T-cell activation-induced gene expression, e.g. for IFN-γ, TNF-α, IL-2 (interleukin-2) and IL-4 genes.

WO 2007/000234 describes the use of fluoroquinolones for treating immune system functional disturbance, e.g. treating and/or preventing immunosuppression. The immune reaction in immunosuppresed mammals is reactivated by administering fluoroquinolones, particularly moxifloxacin, by activation of different immunological cells.

US 2006/004076 discloses combination therapies by using dihydro-epiandrosterone and a fluoroquinolone. WO 2009/009135 teaches specific formulations for topical treatment of psoriasis. WO 2009/023943 discloses topical formulations comprising a quinolone for treatment of bacterial infections.

Most of the available anti-inflammatory pharmaceutical therapies comprise corticosteroids or NSAIDs (non steroidal anti-inflammatory drugs) having serious drawbacks and/or side effects, and target different steps of the inflammatory cascade. Therapies of chronically inflammatory skin diseases include the use of topical corticosteroid ointments, creams, or injections. Disadvantages of dermal topic application of steroid creams are for example stretch marks and thinning of the skin. There is a strong medical need for novel potent anti-inflammatory compounds or compositions for the treatment and/or prophylaxis, in particularly the dermatological treatment (including skin care), of various indications or conditions where inflammatory diseases and conditions play a role. One object of the invention is to provide an effective medicament for the treatment of inflammatory diseases, such as chronically inflammatory diseases, allergic inflammatory diseases, transplantation-rejection, and inflammatory skin diseases (e.g. psoriasis and atopic dermatitis).

T-cells are activated upon triggering of the T-cell receptor (TCR) by antigen presenting cells. T-cell activation induces inter alia proliferation and differentiation of naive, resting T-cells into different classes of effectors. These processes are governed by autocrine and paracrine actions of proteins secreted by activated lymphocytes. Amongst them, interleukines such as IL-2 and IL-4 are of major importance. IL-2, an antigen-non-specific proliferation factor for T-cells, induces cell cycle progression in resting T-cells and clonal expansion of activated T-cells. Being produced mainly by Th1 effector helper T-cells, IL-2 also plays an important role in shaping the immune response. Differentiation of resting, naive T-cells into Th2 effector helper T-cells is driven by IL-4. Furthermore, IL-4 produced by Th2 cells has a crucial role in humoral immunity. It promotes B-cell activation and isotype switching to IgG1 and IgE 2. Thus, IL-4 production plays an important role in the pathogenesis of allergic inflammation. In atopic dermatitis, a chronic allergic skin disease, elevated levels of IL-4 coincide with increased IgE levels mediating hypersensitivity reactions.

It now was found that the expression of the activation-induced T-cell death (AICD) mediator, CD95 (Apo-1, Fas) ligand (L), in pre-activated T-cells, can only be triggered by the simultaneous presence of an IP3/Iono-introduced Ca²⁺ signal and a DAG/PMA-introduced hydrogen peroxide (H₂O₂) signal. Neither second messenger is sufficient by itself. Moreover, it was observed that the H₂O₂-mediated oxidative signal results from PKCθ-dependent production of ROS by the mitochondrial electron transport chain (ETC) respiratory complex I (NADH: ubiquinone oxidoreductase). A new biochemical signalling pathway in T-cells was found showing that mitochondria-generated reactive oxygen species (ROS) play a key role in activation-induced cell death of T-cells in (AICD) by transcriptional regulation of CD95L (ligand) and is further essential for the production of the T-cell activation-related cytokines, IL-2 and IL-4.

The oxidative signal (hydrogen peroxide, (H₂O₂) produced upon T-cell receptor triggering, combined with simultaneous calcium (Ca²⁺) influx into the cytosol is the minimal requirement for induction of CD95L expression. Mitochondrial respiratory complex I (NADH-quinone oxidoreductase) is the molecular source of T-cell activation-induced ROS. In concordance with the regulatory principle identified for CD95L expression, IL-2 and IL-4 expression requires a synergistic action of the Ca²⁺ signal and the mitochondrial complex 1-generated H₂O₂ signal.

It is known from literature that ciprofloxacin treatment may have diverse immune-modulatory effects on T-cells. For example in the publication Stünkel et al. (Clin Exp Immunol. 1991; 86(3); 525-31) it is shown that short-term (up to 96 hours) or simultaneous ciprofloxacin treatment enhanced proliferation of PHA-activated human T-cells and IL-2 secretion.

It surprisingly turned out, that prolonged, long-term treatment with fluoroquinolones, such as ciprofloxacin, of pre-activated human T-cells leads to loss of the mtDNA content and thus exerts immunosuppressive effects on human T-cells suppressing the above described novel mechanism. This is accompanied by impaired activity of the mtDNA-encoded mitochondrial enzymes, such as mitochondrial respiratory complex I, whereas the activities of the nuclear-coded mitochondrial enzymes, complex II (succinate dehydrogenase) and citrate synthase are unaffected. In addition, prolonged ciprofloxacin treatment results in a dose-dependent inhibition of the T-cell activation-induced oxidative signal as well as IL-2 and IL-4 gene expression. Therefore, the fluoroquinolones (and compositions comprising them) for the treatment and or prophylaxis of T-cell mediated inflammatory disorders are important aspects of the invention.

It was found that sustained treatment of pre-activated primary human T-cells with ciprofloxacin results in a dose-dependent inhibition of T-cell receptor (TCR)-induced generation of reactive oxygen species (ROS) and IL-2 and IL-4 expression. This is accompanied by loss of mitochondrial DNA and a resulting decrease in activity of the complex I. Consequently, using a complex I inhibitor or siRNA-mediated down-regulation of the complex I chaperone NDUFAF1, it was found that TCR-triggered ROS generation by complex I is important for activation-induced and IL-4 expression in resting and preactivated human T-cells. This oxidative signal (H₂O₂) synergizes with a Ca²⁺ influx for IL-2/IL-4 expression and facilitates the induction of the transcription factors NF-κB and in AP-1. Moreover, using T-cells isolated from patients with atopic dermatitis, it could be demonstrated that inhibition of complex I-mediated ROS generation blocks disease-associated spontaneous and TCR-induced hyper-expression of IL-4. Thus, it was found that the activation phenotype of T-cells is controlled by a mitochondrial complex I-originated oxidative signal.

Furthermore, by using various experimental models, like ethidium bromide-induced mtDNA depletion, inhibition of complex I or siRNA-mediated knock-down of the complex I chaperone NDUFAF1, it was demonstrated that TCR-triggered ROS generation by the mitochondrial complex I is important for T-cell activation-induced IL-2 and IL-4 expression in both resting and pre-activated human T-cells. IL-2 and IL-4 expression requires a synergistic action of the Ca²⁺ signal and the mitochondrial complex I-derived oxidative signal in the form of H₂O₂. The oxidative signal facilitates activation of redox-dependent transcription factors NF-κB and AP-1 (crucial transcription factors for the activation-induced expression of IL-2, IL-4 and CD95L). Moreover, using T-cells isolated from patients with atopic dermatitis, it could be shown that the inhibition of mitochondrial complex I leads to a significant decrease in spontaneous and TCR-induced IL-4 hyperexpression. Thus, it turned out that mitochondrial complex I-derived ROS controls T-cell activation. Blocking mitochondrial ROS generation or application of prolonged ciprofloxacin treatment opens new possibilities for the treatment of allergic inflammation and Th2-mediated diseases.

Disease states such as acute lung injury (ALI), acute respiratory distress syndrome (ARDS), asthma bronchiale (asthma), chronic obstructive pulmonary disease (COPD), psoriasis, rheumatoid arthritis, and sepsis are all associated with tissue inflammation induced and perpetuated by pathologically activated leukocytes infiltrating the respective tissue.

In addition, exaggerated leukocyte infiltration contributes to the pathogenesis of ischemic-reperfusion injury (IRI) associated with organ transplantation, cardiopulmonary bypass or percutaneous transluminal angioplasty.

The present invention is directed to a pharmaceutical composition comprising at least one compound of formula (I)

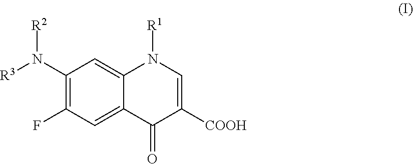

Wherein:
R¹ denotes hydrogen, straight chain C₁-C₆-alkyl, branched C₃-C₆-alkyl, C₃-C₆-cycloalkyl, C₂-C₆-alkenyl, C₂-C₆-alkinyl, all of which may be substituted by one, two or more radicals selected from hydroxyl, C₁-C₆-alkoxy, C₁-C₆-alkylmercapto, and C₁-C₆-alkoxycarbonyl,
R² and R³ are identical or different and denote hydrogen, straight chain C₁-C₆-alkyl, branched C₃-C₆-alkyl, C₃-C₆-cycloalkyl, C₂-C₆-alkenyl, C₁-C₆-alkinyl, all of which may be substituted by one, two or more radicals selected from hydroxyl, C₁-C₆-alkoxy alkoxy, C₁-C₆-alkylmercapto, and C₁-C₆-alkoxycarbonyl,
or R² and R³ together with the nitrogen atom carrying them form a 3 to 7 membered carboxylic ring which can be interrupted by one or more further hetero-atom selected from N, O, and S, and which may be substituted by one, two or more radicals selected from hydroxyl, C₁-C₆-alkoxy, C₁-C₆-alkylmercapto, and C₁-C₆-alkoxycarbonyl,
   or a pharmaceutically acceptable salt, [ester or amide] or an isomeric or polymorphic form thereof and at least one pharmaceutically acceptable excipient (auxiliary) for use as a medicament for the treatment and/or prophylaxis of inflammatory diseases and/or conditions, in particular T-cell mediated inflammatory diseases or conditions.

Furthermore, the present invention relates to the use of a compound of formula (I) as described, a pharmaceutically acceptable salt, [ester or amide] or an isomeric or polymorphic form thereof, for the preparation of a pharmaceutical composition for the treatment and/or prophylaxis of inflammatory diseases or conditions, in particular T-cell mediated inflammatory diseases or conditions.

Preferably R¹ denotes straight chain C₁-C₆-alkyl or C₃-C₆-cycloalkyl. In a preferred embodiment R² and R³ together with the nitrogen atom carrying them form a 3 to 7 membered ring which is interrupted by at least one further hetero-atom selected from N, O, to and S and which can be substituted by one radical selected from hydroxyl, alkoxy, alkylmercapto, and alkoxycarbonyl, more preferred R² and R³ together with the nitrogen atom carrying them form a piperazine ring.

A preferred embodiment is directed to use of compounds of formula (II)

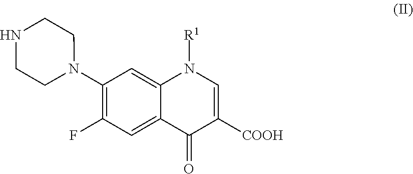

wherein:
R¹ denotes hydrogen, straight chain C₁-C₆-alkyl, branched C₃-C₆-alkyl, C₃-C₆-cycloalkyl, C₂-C₆-alkenyl, C₂-C₆-alkinyl, all of which may be substituted by a radical selected from hydroxyl, C₁-C₆-alkoxy, C₁-C₆-alkylmercapto, and C₁-C₆-alkoxycarbonyl.

In a preferred embodiment the present invention is directed to the use of a compound of formula (II), wherein R¹ denotes ethyl or cyclopropane. Preference is given to the compounds 1-cyclopropyl-6-fluoro-1,4-dihydro-4-oxo-7-(1-piperazinyl)-3-quinoline-carboxylic acid (ciprofloxacin) and 1-ethyl-6-fluoro-1,4-dihydro-4-oxo-7-(1-piperazinyl)-3-quinolinecarboxylic acid (norfloxacin), and the appropriate salts thereof.

A preferred embodiment of the invention is directed to pharmaceutical composition comprising 1-cyclopropyl-6-fluoro-1,4-dihydro-4-oxo-7-(1-piperazinyl)-3-quinoline carboxylic acid (ciprofloxacin), a pharmaceutically acceptable salt, ester or amide or an isomeric or polymorphic form thereof and at least one pharmaceutically acceptable excipient for use as a medicament for the treatment and/or prophylaxis of inflammatory diseases or conditions, in particular T-cell mediated inflammatory diseases or conditions.

The present invention also is directed to the use of stereoisomeric forms of compounds of formula (I) or (II), e.g. pure enantiomeres and mixtures of enantiomeres and racemates of enantiomeres.

The term “pharmaceutical” includes also prophylactic applications in order to prevent to medical conditions where T-cell mediated processes play a role. The term “pharmaceutical” includes also applications, where compounds of the present invention may be used as vehicles for drug targeting of diagnostics or therapeutics.

The pharmaceutical composition may in principle also comprise a prodrug of a compound of formula (I). The term “prodrug” refers to one or more compounds that are rapidly transformed in vitro and from a non-active to active state in vivo to yield the parent compound of the above formulas (I) or (II), for example by hydrolysis in blood or in vivo metabolism.

The term “pharmaceutically acceptable salts, esters, and amides” as used herein refers to those carboxylate salts, amino acid addition salts, esters, and amides of the compounds of the present invention which are, within the scope of sound medical judgment, suitable for use in contact with tissues of patients without undue toxicity, irritation, allergic response and the like, commensurate with a reasonable benefit/risk ratio, and effective for their intended use, as well as the zwitterionic forms, where possible, of the compounds of the present invention.

The term “salts” refers to the relatively non-toxic, inorganic and organic acid addition salts of the compounds of the present invention. These salts can be prepared in situ during the final isolation and purification of the compounds or by separately reacting the purified compounds in its free form with a suitable inorganic or organic acid or base and isolating the salt thus formed. Representative salts of the compounds of the present invention include the hydrobromide, hydrochloride, sulfate, bisulfate, nitrate, acetate, oxalate, valerate, palmitate, stearate, laurate, borate, benzoate, lactate, phosphate, tosylate, citrate, maleate, fumarate, succinate, tartrate, naphthylate, mesylate, glucoheptonate, lactiobionate, laurylsulphonate salts and the like.

These may include cations based on the alkali and alkaline-earth metals, such as sodium, lithium, potassium, calcium, magnesium and the like, as well as non-toxic ammonium, quaternary ammonium and amine cations including, but not limited to, ammonium, tetramethylammonium, tetraethylammonium, methylamine, dimethylamine, trimethylamine, triethylamine, ethylamine, and the like.

Examples of the pharmaceutically acceptable, non-toxic esters of the compounds of this invention include C₁, C₂, C₃, C₄, C₅ and C₆ alkyl esters wherein the alkyl group is a straight or branched chain. Acceptable esters also include C₅, C₆ and C₇ cycloalkyl esters as well arylalkyl esters such as, but not limited to benzyl, C₁, C₂, C₃, C₄, C₅ and C₆ alkyl ester are to preferred. Esters of the compounds of the present invention may be prepared according to conventional methods. Examples of pharmaceutically acceptable, non-toxic amides of compounds of this invention include amides derived from ammonia, primary C₁, C₂, C₃, C₄, C₅ and C₆ alkyl amines and secondary C₁, C₂, C₃, C₄, C₅ and C₆ dialkyl amines wherein the alkyl groups are straight or branched chains. In the case of secondary amines the amine may also be in the form of a 5 or 6 membered heterocycle containing one nitrogen atom. Amides derived from ammonia, C₁, C₂ and C₃ alkyl primary amides and C₁ to C₂ dialkyl secondary amides are preferred. Amides of the compounds of the present invention may be prepared according to conventional methods.

The invention also provides with pharmaceutical, dermatological and/or cosmetic compositions comprising one or several compounds of formula (I) and/or (II). A further preferred embodiment of the invention provides pharmaceutical, dermatological and/or cosmetic compositions comprising at least one compound selected from norfloxacin and ciprofloxacin.

The present invention further provides a method of modulating the T-cell activation induced IL-2 and IL-4 expression due to decreased activity of mitochondrial complex I and activation induced oxidative signal, comprising the step of administering to a patient an effective amount of at least one compound having the structure of formula (I). It has been found that prolonged treatment with compounds of formula (I) leads to mtDNA loss in pre-activated primary human T-cells, consequently to a decrease in activity of mitochondrial complex I and activation-induced oxidative signal. Prolonged treatment with compounds of formula (I) showed clear immunosuppressive effects on the function of pre-activated primary human T-cells.

The invention particularly relates to a pharmaceutical composition comprising at least one compound, a pharmaceutically acceptable salt, or an isomeric or polymorphic form thereof according to formula (I), preferably of formula (III), more preferably of 1-cyclopropyl-6-fluoro-1,4-dihydro-4-oxo-7-(1-piperazinyl)-3-quinoline carboxylic acid (ciprofloxacin), wherein the composition is administered to a mammal, particularly a human, in a prolonged treatment of at least five days, preferably at least 7 days, more preferably at least 10 days, often at least 14 days.

The total daily dosage of the compound of formula (I) according to this invention, administered in single or divided doses, may be in the range up to 50 mg per kilogram of a body weight, preferably up to 10 mg per kilogram of body weight, more preferably up to 5 mg per kilogram of body weight.

In one embodiment of the invention the pharmaceutical composition according as described in the application is administered in a prolonged treatment of at least five days, preferably at least 7 days, more preferably at least 10 days, often at least 14 days, in an daily dosage in the range of 0.1 to 5 mg per kilogram of body weight, preferably of 0.1 to 1.5 mg per kilogram of body weight, more preferably in the range of 0.1 to 0.5 mg per kilogram of body weight, particularly in the range of 0.1 to 0.125 5 mg per kilogram of body weight.

In a further preferred embodiment of the invention, the pharmaceutical composition is administered as described above, in a prolonged topical medication, preferably to the skin, e.g. as a transdermal medication of at least five days, preferably at least 7 days, more preferably at least 10 days, often at least 14 days. Dosage unit compositions may contain such submultiples thereof as may be used to make up the daily dosage. It will be understood, however, that the specific dose level for any particular patient, whether human or other animal, will depend upon a variety of factors including the body weight, general health, sex, diet, time and route of administration, rates of absorption and excretion, combination with other drugs and the severity of the particular disease being treated.

The pharmaceutical composition containing compound (I) as described above may be administered for example from 3 to 5 days, preferably from 3 to 7 days, more preferably from 3 to 10 days, often at least 14 days, each day in a daily dosage in the range of 0.1 to 5 mg per kilogram of body weight, preferably of 0.1 to 1.5 mg per kilogram of body weight, more preferably in the range of 0.1 to 0.5 mg per kilogram of body weight, particularly in the range of 0.1 to 0.125 5 mg per kilogram of body weight.

Pharmaceutically compositions of the present invention comprise a pharmaceutically acceptable carrier and at least one compound of formula (I), whereby a pharmaceutically acceptable carrier can also be a medically appropriate nano-particle, dendrimer, liposome, microbubble or polyethylene glycol (PEG). The pharmaceutical compositions of the present invention may include one or more of the compounds having the above structure (I) formulated together with one or more, physiologically acceptable carriers, adjuvants or vehicles, which are collectively referred to herein as carriers, for parenteral injection, for oral administration in solid or liquid form, for rectal or topical administration, topical application to skin and/or mucosa, transdermal application and the like. The compositions can be administered to humans (and animals) either orally, rectally, parenterally (such intravenously, intramuscularly, intradermally or subcutaneously), intracisternally, intravaginally, interperitoneally, locally (powders, ointments or drops), or as a buccal or by inhalation (nebulized, or as nasal sprays). Preferably, the pharmaceutical composition as described in the present invention is made to be administered via a parenteral route, in particular for dermal treatment.

In a preferred embodiment the composition described in the present invention is administered in a dermal topical medication, selected from emulsion, solution, suspension, lotion, shake lotion, cream, ointment, gel, foam, and transdermal patch (e.g. TTS). Preferably, the composition comprising compound (I) is administered in a topical medication for treatment of inflammatory skin diseases or conditions.

In a preferred embodiment the composition described in the present invention is administered to a mammal, particularly a human, in a dermal topical medication as mentioned above, wherein the composition is administered in a prolonged treatment of at least 5 days, preferably at least 7 days, more preferably at least 10 days, often at least 14 days, and wherein the active compound according to Formula (I), in particular 1-cyclopropyl-6-fluoro-1,4-dihydro-4-oxo-7-(1-piperazinyl)-3-quinolinecarboxylic acid (ciprofloxacin) may be administered in an amount representing from 0.001% to 40% by weight, preferentially 0.005% to 30% by weight and more preferentially from 0.01% to 20% by weight, more preferentially from 0.001% to 10% by weight (referring to total composition).

Excipients and further additives of compositions suitable for topical-dermal application are the similar to those described later concerning dermatological/cosmetic composition.

In another embodiment the composition described in the present invention is administered by parenteral medication selected from intramuscularly injection, intradermally injection and subcutaneously injection.

Compositions suitable for parenteral injection may comprise physiologically acceptable sterile aqueous or nonaqueous solutions, stabilizers, antioxidants, preservatives (e.g. ascorbic acid, sodium sulfite, sodium hydrogen sulfite, benzyl alcohol, EDTA), dispersions, suspensions or emulsions and sterile powders for reconstitution into sterile injectable solution or dispersion. Examples of suitable aqueous and nonaqueous carriers, diluents, solvents or vehicles include water, ethanol, polyol, (propylene glycol, polyethylene glycol, glycerol and the like), suitable mixtures thereof, vegetable oils (such as olive or canola oil) and injectable organic esters such as ethyl oleate. Proper fluidity can be maintained, for examples, by the use of a coating such as lecithin, by the maintenance of the required particle size in the case of dispersions and by the use of surfactants.

These compositions may also contain adjuvants such as preserving, wetting, emulsifying, and dispersing agents. Prevention of the actions of microorganisms can be ensured by various antibacterial and antifungal agents, for examples, parabens, chlorobutanol, phenol, sorbic acid, and the like. It may also be desirable to include isotonic agents, for examples sugars, sodium chloride and the like. Prolonged absorption of the injectable pharmaceutical form can be brought about by the use of agents delaying absorption, for examples aluminium monostearate and gelatin. If desired, and for more effective distribution, the compounds can be incorporated into slow or timed release or targeted delivery systems such as polymer matrices, liposomes, and microspheres. They may be sterilized, for example, by filtration through a bacteria-retaining filter, or by incorporating sterilizing agents in the form of sterile water, or some other sterile injectable medium immediately before use.

The pharmaceutical composition can also be administered orally. Solid dosage forms for oral administration include capsules, tablets, pills, powders and granules. In such solid dosage forms, the active compound or a prodrug is admixed with at least one inert customary excipient (or carrier) such as sodium citrate or dicalcium phosphate or (i) fillers or extenders, as for example, starches, lactose, sucrose, glucose, mannitol and silicic acid, (ii) binders, as for example, carboxymethylcellulose, alginates, gelatine, polyvinylpyrrolidone, sucrose and acacia, (iii) humectants, as for example, glycerol, (div disintegrating agents, as for example, agar-agar, calcium carbonate, potato or tapioca starch, aliginic acid, certain complex silicates and sodium carbonate, (v) solution retarders, as for examples, paraffin, (vi) absorption accelerators, as for example, quaternary ammonium compounds, (vii) wetting agents, as for examples, cetyl alcohol and glycerol monostearate, (viii) adsorbents, as for example, kaolin and bentonite, and (ix) lubricants, as for example, talc, calcium stearate, magnesium stearate, solid polyethylene glycols, sodium lauryl sulfate and mixtures thereof.

In the case of capsules, tablets, and pills, the dosage forms may also comprise buffering agents. Solid compositions of a similar type may also be employed as fillers in soft and hard-filled gelatine capsules using excipients as lactose or milk sugars as well as high molecular polyethylene glycols and the like. Solid dosage forms such as tablets, dragées, capsules, pills and granules can be prepared with coatings and shells, such as enteric coatings and others well known in the art. They may contain opacifying agents, and can also be of such compositions that they release the active compound or compounds in a certain part of the intestinal tract in a delayed manner. Examples of embedding compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, cannola oil, caster oil and sesame seed oil, glycerol, tetrahydrofurfuryl alcohol, polyethylene glycols and fatty acid esters of sorbitan or mixtures of these substances, and the like. Besides such inert diluents, the compositions can also include adjuvants, such as wetting agents, emulsifying and suspending agents, sweeting, flavouring and perfuming agents. Suspensions, in addition to the active compounds, may contain suspending agents, for example, ethoxylated isostearyl alcohols, polyoxyethylene sorbitol and sorbitan esters, microcrystalline cellulose, aluminium metahydroxide, bentonite, agar-agar, tragacanth or mixtures of these substances and the like.

Compositions for rectal administrations are preferably suppositories, which can be prepared by mixing the compounds of the present invention with suitable nonirritating excipients or carriers such as cacao butter, polyethylene glycol or a suppository wax, which are solid at ordinary temperatures but liquid at body temperature and therefore melt in the rectal or vaginal cavity and release the active component. Dosage forms for topical administration of a compound of this invention include ointments, powder, sprays and inhalants. The active component of formula (I) is admixed under sterile conditions with a physiologically acceptable carrier and any needed preservatives, buffers or propellants as may be required. Ophthalmic formulations, eye ointments, suspensions, powder and solutions are also contemplated as being within the scope of this invention.

The compounds of formula (I) can also be incorporated into or connected to liposomes or administrated in the form of liposomes. As is known in the art, liposomes are generally derived from phospholipids or other lipid substances. Liposomes are formed by mono or multilamellar hydrated liquid crystals that are dispersed in an aqueous medium. Any non-toxic, physiologically acceptable metabolized lipid capable of forming liposomes can be used. The present compositions in liposome form can contain, in addition to the selectin binding antagonists of the present invention, stabilizers, preservatives, excipients and the like. The preferred lipids are the phospholipids and the phosphatidyl cholines (lecithins), both natural and synthetic. Methods to form liposomes are well known in the art.

Non-parenteral dosage forms may also contain a bioavailability enhancing agent (e.g. enzyme modulators, antioxidants) appropriate for the protection of the compounds against degradation. Actual dosage levels of active ingredient in the composition of the present invention may be varied so as to obtain an amount of active ingredient that is effective to obtain the desired therapeutic response for a particular composition and method of administration. The selected dosage level, therefore, depends on the desired therapeutic effect, on the route of administration, on the desired duration of treatment and other factors. The total daily dosage of the compounds on this invention administered to a host in single or divided doses may be in the range up to 50 mg per kilogram of body weight. Dosage unit compositions may contain such submultiples thereof as may be used to make up the daily dosage. It will be understood, however, that the specific dose level for any particular patient, whether human or other animal, will depend upon a variety of factors including the body weight, general health, sex diet, time and route of administration, rates of absorption and excretion, combination with other drugs and the severity of the particular disease being treated.

Further, the present invention relates to dermatological or cosmetic compositions comprising at least one compound of formula (I), preferably 1-cyclopropyl-6-fluoro-1,4-dihydro-4-oxo-7-(1-piperazinyl)-3-quinoline carboxylic acid (ciprofloxacin), a pharmaceutically acceptable salt, ester or amide or an isomeric or polymorphic form thereof and at least one dermatological or cosmetic tolerable component, e.g. a dermatological or cosmetically tolerable component for skin applications. Dermatologically tolerable components that can be used for the dermatological compositions described here are identical to the cosmetically tolerable components as defined in this invention. In addition to dermatologically inactive ingredients the dermatological compositions may also comprise dermatologically or pharmaceutically active ingredients.

The amount used of compound of formula (I), a salt, ester or amide of compound of formula (I), or an isomeric or polymorphic form thereof corresponds to the amount required to obtain the desired result using the dermatological or cosmetic compositions.

One skilled in this art is capable of evaluating this effective amount, which depends on the derivative used, the individual on whom it is applied, and the time of this application. To provide an order of magnitude, in the cosmetic or dermatological compositions according to the invention, the compound of formula (I), a salt of compound of formula (I) or an isomeric or polymorphic form thereof may be administered in an amount representing from 0.001% to 40% by weight, preferentially 0.005% to 30% by weight and more preferentially from 0.01% to 20% by weight. The amounts of the various components of the physiological medium of the pharmaceutical, dermatological or cosmetic composition according to the invention are those generally included in the fields under consideration.

When the dermatological or cosmetic composition is an emulsion, the proportion of the fatty phase may range from 2% to 80% by weight and preferably from 5% to 50% by weight relative to the total weight of the cosmetic composition. The aqueous phase may be adjusted as a function of the content, in the fatty phase, of compound of formula (I), a salt, ester or amide of compound of formula (I) or an isomeric or polymorphic form thereof, of alcohol and nonionic surfactant and also that of the optional additional ingredients, to obtain 100% by weight. In practice, the aqueous phase represents from 5% to 90% by weight. The fatty phase may contain fatty or oily compounds, which are liquid at room temperature (25° C.), generally known as oils, waxes and pasty or semi-solid products. These oils may be mutually compatible and may form a macroscopically homogeneous liquid fatty phase. The aqueous phase contains water and optionally a water-miscible ingredient, for instance polyols such as propylene glycol, glycerol or sorbitol.

In particular, the dermatological or cosmetic composition often contains one or more components such as nonionic surfactants. The nonionic surfactant or the mixture of nonionic surfactants with a hydrophilic-lipophilic balance (HLB) of greater than 10 is (are) preferably used in an amount sufficient to dissolve with the compound of formula (I), a salt, ester or amide, or an isomeric or polymorphic form thereof.

In practice, this nonionic surfactant or mixture of nonionic surfactants may be included in the compositions of the invention in a concentration ranging from 0.01% to 10% by weight, preferentially from 0.05% to 5% by weight and more preferentially from 0.1% to 2% by weight.

More specifically, dermatological or cosmetic compositions of the present invention contain nonionic surfactants having a HLB of greater than 10 and which may be up to 20. These are compounds that are well known per se [see Handbook of Surfactants by M. R. Porter, published by Blackie & Son (Glasgow and London), 1991, pp. 116-178]. Thus, they may be selected especially from among polyethoxylated, polypropoxylated or polyglycerolated fatty acids, (C1-C20) alkylphenols, alpha-diols and alcohols, which are preferably hydrogenated, with a fatty chain containing, for example, front 8 to 22 carbon atoms, the mean number of ethylene oxide or propylene oxide structural units optionally ranging especially from 3.5 to 200 (for example from 5 to 100) and the number of glycerol to groups optionally ranging especially from 2 to 100 (for example from 3 to 50), and mixtures thereof. Also exemplary are copolymers of ethylene oxide and propylene oxide, condensates of ethylene oxide and propylene oxide on fatty alcohols; polyethoxylated fatty amides and preferably those containing on average from 3.5 to 200 mol of propylene oxide and/or ethylene oxide; polyglycerolated fatty amides and preferably those containing on average from 1.5 to 40; ethoxylated fatty acid esters of sorbitan especially containing from 2 to 30 mol on average of ethylene oxide and a fatty chain especially containing from 8 to 22 (for example from 12 to 18) carbon atoms; fatty acid esters of sucrose; fatty acid esters of polyethylene glycol; (C6-C24) alkylpolyglycosides; N—(C6-C24) alkylglucaminc derivatives; amine oxides such as (C10-C14) alkylamine oxides; and mixtures thereof.

In the dermatological or cosmetic compositions according to the present invention an alcohol may be used alone or as a mixture and is selected from among C1-C4 alcohols such as ethanol or isopropanol, and mixtures thereof. The C1-C4 alcohol is preferably included in an amount sufficient to dissolve with the nonionic surfactant the compound of formula (I) or an isomeric or polymorphic form thereof. In practice, the C1-C4 alcohol or the mixture of C1-C4 alcohols may be included in the cosmetic compositions of the invention in a concentration ranging from 2% to 80% of the total weight of the composition, preferentially from 10% to 70% and more preferentially from 20% to 60% by weight relative to the total weight of the cosmetic composition.

The dermatological or cosmetic compositions of the invention are preferentially for topical application to the skin of animals (including humans).

Thus, the dermatological or cosmetic compositions should contain a non-toxic physiologically acceptable medium that can be applied to human skin. For a topical application to the skin, the composition may be in the form of a solution, a suspension, an emulsion or a dispersion of more or less fluid consistency and especially liquid or semi-liquid consistency, obtained by dispersing a fatty phase in an aqueous phase (O/W) or, conversely, (W/O), or alternatively a gel.

A cosmetic composition in the form of a mousse or in the form of a spray or an aerosol then comprising a pressurized propellant may also be provided. Also the compositions may be in the form of a haircare lotion, a shampoo or hair conditioner, a liquid or solid soap, a treating mask, or a foaming cream or gel for cleansing the hair. They may also be in the form of a hair dye or hair mascara.

In particular, the present invention relates to a pharmaceutical, dermatological or cosmetic composition as described above wherein the pharmaceutical composition is administered in a dermal topical medication selected from emulsion, solution, suspension, lotion, shake lotion, creame, ointment, gel, foam, and transdermal patch (TTS) and wherein the to inflammation diseases or conditions are inflammatory skin diseases or conditions.

The pharmaceutical, dermatological or cosmetic compositions of the invention may also comprise one or more other ingredients usually employed in the fields under consideration, selected from among formulation additives, for instance aqueous-phase or oily-phase thickeners or gelling agents, dyestuffs that are soluble in the medium of the cosmetic composition, solid particles such as mineral or organic fillers or pigments in the form of microparticles or nanoparticles, preservatives, fragrances, hydrotopes or electrolytes, neutralizers (acidifying or basifying agents), propellants, anionic, cationic or amphoteric surfactants, polymers, in particular water-soluble or water-dispersible anionic, nonionic, cationic or amphoteric film-forming polymers, mineral or organic salts, chelating agents; mixtures thereof.

These additives may be present in the cosmetic composition in the amounts generally employed in cosmetics, and especially in a proportion of from 0.01% to 50% and preferably from 0.1% to 20%, for example from 0.1% to 10%, of the total weight of the cosmetic composition.

The present invention also relates to pharmaceutical, dermatological or cosmetic compositions comprising a compound of formula (I), a pharmaceutically acceptable salt, (ester, or amide) of compound of formula (I) or an isomeric or polymorphic form thereof and at least one further pharmaceutically, dermatologically or cosmetically active ingredient. The pharmaceutically, dermatologically or cosmetically active ingredients that can be used for the compositions described in the following.

As cosmetically/dermatologically/pharmaceutically active agents with beneficial action the following are cited: of active agents selected from among: UV-blocking agents, such as sunscreens; vitamins (A, C or E) and derivatives thereof (retinyl palmitate, tocopheryl acetate or tocopheryl palmitate); ceramides; proteins and protein hydrolysates, peptides and amino acids; urea and allantoin;

sugars and sugar derivatives, for instance reduced or oxidized sugars; extracts of plant origin (those from Iridacea plants or from soybean) or of bacterial origin; hydroxy acids, in particular hydroxycarboxylic acids or ketocarboxylic acids (fruit acid, salicylic acid) and esters thereof, for instance 5-n-octanoylsalicylic acid; diazoxide, spiroxazone, or phospholipids, for instance lecithin; anti-bacterial, anti-fungal or anti-dandruff agents, for instance selenium derivatives, ketoconazole, octopirox, triclocarban, triclosan, zinc pyrithione, itraconazole, asiatic acid, hinokitiol, mipirocine, tetracyclines, especially erythromycin, benzoyl peroxide or benzyl peroxide and minocycline: calcium-channel antagonists and potassium-channel agonists; hormones; steroidal anti-inflammatory agents, for instance glucocorticoids, corticosteroids (for example: hydrocortisone) and non-steroidal anti-inflammatory agents, for instance glycyrrhetinic acid and [alpha]-bisabolol, benzydamine and the compounds described in EP-A 0 770 399 and WO 1994/06434; antioxidants, for instance butylhydroxyanisole or butylhydroxytoluene; anti-seborrhoeic agents; anti-parasitic agents; anti-viral agents; anti-pruriginous agents; carotenoids, for instance [beta]-carotene; lactones and the corresponding salts thereof, essential fatty acids, for instance linoleic acid, eicosatetraenoic acid, linolenic acid and eicosatrienoic acid, or esters and amides thereof; essential oils; phenols and polyphenols, for instance flavonoids; and mixtures thereof. This additional active agent may be for example in an amount of from 0.001% to 10% by weight, preferably from 0.1% to 5% and better still from 0.5% to 3% by weight.

A further aspect of this invention deals with a process for the preparation of a pharmacologic, dermatologic or cosmetical composition by mixing a compound of formula (I), preferably 1-cyclopropyl-6-fluoro-1,4-dihydro-4-oxo-7-(1-piperazinyl)-3-quinoline carboxylic acid (ciprofloxacin), a pharmaceutically acceptable salt, ester or amide or an isomeric or polymorphic form thereof and at least one pharmaceutically/dermatologically/cosmetically tolerable component and eventually further pharmaceutically active ingredients.

The invention in a particular embodiment relates to a pharmaceutical composition, which comprises 1-cyclopropyl-6-fluoro-1,4-dihydro-4-oxo-7-(1-piperazinyl)-3-quinoline carboxylic acid (ciprofloxacin) or a pharmaceutically acceptable salt, (ester or amide) or an isomeric or polymorphic form thereof.

The invention in a further embodiment relates to a pharmaceutical composition for use as a medicament, wherein the composition is administered in a prolonged treatment of at least five days. The invention in a particular embodiment relates to a pharmaceutical composition for use as a medicament, wherein the composition is administered in a prolonged treatment of at least five days in an daily dosage in the range of 0.1 to 1.5 mg/kg body weight. The pharmaceutical composition often is administered in a prolonged topical dermal application of at least 5 days.

The invention in a particular embodiment relates to a pharmaceutical composition for use as a medicament, wherein the pharmaceutical composition is administered in a topical dermal application and wherein the composition is selected from emulsion, solution, suspension, lotion, shake lotion, cream, ointment, gel, foam, and transdermal patch and wherein the inflammation diseases or conditions are inflammatory skin diseases or conditions. The invention in a particular embodiment relates to a pharmaceutical composition for use as a medicament, wherein the inflammatory discuses or conditions are allergic inflammation diseases or conditions. The invention in a particular embodiment relates to a pharmaceutical composition for use as a medicament, wherein the inflammatory diseases or conditions are T-cell mediated inflammatory diseases or conditions.

The pharmaceutical composition can be used as a medicament for inflammatory diseases or conditions selected from:

asthma bronchiale, psoriasis, atopic dermatitis, systemic lupus erythematosus (SLE), Sjörgen's syndrome, rheumatoid arthritis, encephalitis and in particular acute disseminated encephalomyelitis (ADEM), Addison's disease, antiphospholipid antibody syndrome (APS), aplastic anemia, autoimmune hepatitis, coeliac disease, inflammatory bowel disease and in particular Crohn's disease, diabetes mellitus (type 1), Goodpasture's syndrome, hyperthyroidism and in particular Graves' disease, Guillain-Barré syndrome (GBS; also called acute inflammatory demyelinating polyneuropathy, acute idiopathic polyradiculoneuritis, acute idiopathic polyneuritis and Landry's ascending paralysis), hypothyroidism and in particular Hashimoto's disease, idiopathic thrombocytopenic purpura, lupus erythematosus, multiple sclerosis, myasthenia gravis, opsoclonus myoclonus syndrome (OMS), optic neuritis, thyroiditis and in particular Ord's thyroiditis, pemphigus, polyarthritis, primary biliary cirrhosis, psoriasis, rheumatoid arthritis, Reiter's syndrome, Sjögren's syndrome, Takayasu's arteritis, temporal arteritis, warm autoimmune hemolytic anemia, vasulitis and in particular Wegener's granulomatosis, alopecia universalis, Behcet's disease, Chagas' disease, chronic fatigue syndrome, dysautonomia including postural orthostatic tachycardia syndrome (POTS), endometriosis, hidradenitis suppurativa, interstitial cystitis, neuromyotonia, sarcoidosis, scleroderma, ulcerative colitis, vitiligo, vulvodynia and graft-versus-host.

The invention in a particular embodiment relates to a pharmaceutical composition for use as a medicament, wherein the inflammatory diseases or conditions are inflammatory skin diseases or conditions selected from psoriasis and atopic dermatitis.

In particular, the compounds of formula (I) may be used to treat a variety of diseases relating to inflammation and in particular with T-cell intermediate inflammation diseases to and conditions. For example, the compounds of the present invention may be administrated to a patient to treat (or prevent) Chronic Obstructive Pulmonary Disease (COPD), acute lung injury (ALI), cardiopulmonary bypass, acute respiratory distress syndrome (ARDS). Crohn's disease, septic shock, sepsis, chronic inflammatory diseases such as psoriasis, atopic dermatitis, and rheumatoid arthritis, and reperfusion injury that occurs following heart attacks, strokes, atherosclerosis, and organ transplants, traumatic shock, multi-organ failure, autoimmune diseases like multiple sclerosis, percutaneous transluminal angioplasty, asthma and inflammatory bowel disease. The compounds of formula (I) may also be administrated in the context of transplantation, e.g. to treat Graft-versus-host.

The compounds of formula (I) and compositions comprising them can in particular be used for the treatment and or prophylaxis of T-cell mediated inflammatory disorders from the group Chronic Obstructive Pulmonary Disease (COPD), acute lung injury (ALI), rheumatoid arthritis, lupus erythematosus, reperfusion injury that occurs following organ transplants and asthma. The compounds of formula (I) may also be administrated in the context of transplantation, e.g. to treat Graft-versus-host. They can also be used for diabetes and MS-treatment.

In each case, an effective amount of the compounds of the present invention is administered either alone or as part of a pharmaceutically active composition to a patient in need of such treatment. It is also recognized that a combination of the compounds may be administered to a patient in need of such administration. The compounds of the present invention may also be administered to treat other diseases that are associated with T-cell mediated inflammatory process modulate the IL-2 and IL-4 expression.

The use of the active ingredients according to the invention or of pharmaceutical, cosmetic or dermatological compositions with an effective content of active ingredient according to the invention surprisingly enables effective treatment, but also prophylaxis of inflammatory skin diseases or conditions.

The invention particularly relates to the use of a compound of formula (I), preferably 1-cyclopropyl-6-fluoro-1,4-dihydro-4-oxo-7-(1-piperazinyl)-3-quinoline carboxylic acid (ciprofloxacin), a pharmaceutically acceptable salt, or an isomeric or polymorphic form thereof for the preparation of a cosmetic or dermatological composition.

### BRIEF DESCRIPTION OF THE FIGURES FROM FIG. 1 TO FIG. 8

FIG. 1A shows the loss of mtDNA (mitochondrial DNA) content in pre-activated primary human T-cells after prolonged treatment with 75 μg/ml ciprofloxacin (Cipro) for 7 days. The amount of mtDNA was estimated by gene expression ratio between mitochondrial 12s rRNA gene and nuclear encoded GAPDH. T-cells from at least three healthy donors were analyzed.

FIG. 1B shows the loss of mtDNA (mitochondrial DNA) content in pre-activated primary human T-cells after prolonged treatment with different amounts (25, 50, 75 μg/ml) ciprofloxacin (Cipro) for 7 days. The amount of mtDNA was estimated by gene expression ratio between fragment of heavy strand of mitochondrial origin of replication (mito Ori) and nuclear encoded gene RSP9. T-cells from at least three healthy donors were analyzed.

FIG. 2 shows the impaired activity of mitochondrial respiratory complex I in pre-activated human T-cells after prolonged treatment with different amounts (25, 50, 75 μg/ml) ciprofloxacin (Cipro) for 7 days. The activity of mitochondrial respiratory complex I (NADH-quinone oxidoreductase) was measured by real-time spectrophotometry (15 min, 20° C.).

FIG. 3 shows the blocked activation-induced ROS generation in pre-activated primary human T-cells after prolonged treatment with different amounts (20, 50, 75 μg/ml) ciprofloxacin (Cipro) for 7 days. The pre-activated primary human T-cells were stained with redox-sensitive fluorescent probe DCF-DA (20 min, 37° C.) and subsequently activated via plate-bound agonistic anti-CD3 antibody (30 μg/ml). The level of activation-induced ROS was assessed by FACS measurement. T-cells from at least three healthy donors were analyzed.

FIG. 4A shows basal IL-2 expression in pre-activated primary human T-cells after prolonged treatment with different amounts (20, 50, 70 μg/ml) ciprofloxacin (Cipro) for 7 days. Expression levels of IL-2 was analyzed using quantitative real-time SYBRGreen PCR and were estimated by gene expression ratio between IL-2 gene and GAPDH genes.

FIG. 4B shows basal IL-4 expression in pre-activated primary human T-cells after prolonged treatment with different amounts (20, 50, 70 μg/ml) ciprofloxacin (Cipro) for 7 days. Expression levels of IL-4 was analyzed using quantitative real-time SYBRGreen PCR and were estimated by gene expression ratio between IL-4 gene and GAPDH genes.

FIG. 5A shows immunosuppressive effect in pre-activated primary human T-cells after prolonged treatment with different amounts (20, 50, 70 μg/ml) ciprofloxacin (Cipro) for 7 days. Primary human T-cells were activated with plate-bound anti-CD3 agonistic antibody for 1 h. Expression levels of IL-2 were analyzed using quantitative real-time SYBRGreen PCR and estimated by gene expression ratio between IL-2 gene and GAPDH genes. Data are shown as fold increase of induction of gene expression (anti-CD3 activated cells vs. respective control).

FIG. 5B shows immunosuppressive effect in pre-activated primary human T-cells after prolonged treatment with different amounts (20, 50, 70 μg/ml) ciprofloxacin (Cipro) for 7 days. Primary human T-cells were activated with plate-bound anti-CD3 agonistic antibody for 1 h. Expression levels of IL-4 were analyzed using quantitative real-time SYBRGreen PCR and estimated by gene expression ratio between IL-4 gene and GAPDH genes. Data are shown as fold increase of induction of gene expression (anti-CD3 activated cells vs. respective control).

FIG. 6A shows activation-induced IL-4 expression in pre-activated peripheral human blood T-cells of healthy donors after being cultured in presence, or absence of 50 μg/ml ciprofloxacin (Cipro) for 7 days. The cells were activated via plate-bound anti-CD3 antibody for 1 h. Expression levels of IL-4 were analyzed using quantitative real-time SYBRGreen PCR and estimated by gene expression ratio between IL-4 and GAPDH gene.

FIG. 6B shows activation-induced IL-4 expression in pre-activated peripheral human blood T-cells of patients with atopic dermatitis after being cultured in presence or absence of 50 μg/ml ciprofloxacin (Cipro) for 7 days. The cells were activated via plate-bound anti-CD3 antibody for 1 hour. Expression levels of IL-4 were analyzed using quantitative real-time SYBRGreen PCR and estimated by gene expression ratio between IL-4 and GAPDH gene.

FIG. 6C shows activation-induced IL-2 expression in pre-activated peripheral human blood T-cells of healthy donors after being cultured in presence or absence of 50 μg/ml ciprofloxacin (Cipro) for 7 days. The cells were activated via plate-bound anti-CD3 antibody for 1 hour. Expression levels of IL-2 were analyzed using quantitative real-time SYBRGreen PCR and estimated by gene expression ratio between IL-2 and GAPDH gene.

FIG. 6D shows activation-induced IL-2 expression in pre-activated peripheral human blood T-cells of patients with atopic dermatitis after being cultured in presence or absence of 50 μg/ml ciprofloxacin (Cipro) for 7 days. The cells were activated via plate-bound anti-CD3 antibody for 1 h. Expression levels of IL-2 were analyzed using quantitative real-time to SYBRGreen PCR and estimated by gene expression ratio between IL-2 and GAPDH gene.

FIG. 6E shows activation-induced CD95L expression in pre-activated peripheral human blood T-cells of healthy donors after being cultured in presence or absence of 50 μg/ml ciprofloxacin (Cipro) for 7 days. The cells were activated via plate-bound anti-CD3 antibody for 1 h. Expression levels of CD95L were analyzed using quantitative real-time SYBRGreen PCR and estimated by gene expression ratio between CD95L and GAPDH genes.

FIG. 6F shows activation-induced CD95L expression in pre-activated peripheral human blood T-cells of patients with atopic dermatitis after being cultured in presence or absence of 50 μg/ml ciprofloxacin (Cipro) for 7 days. The cells were activated via plate-bound anti-CD3 antibody for 1 h.

Expression levels of CD95L were analyzed using quantitative real-time SYBRGreen PCR and estimated by gene expression ratio between CD95L and GAPDH genes.

FIG. 7A shows activation-induced IL-4 secretion in pre-activated peripheral blood T cells of two healthy donors (control A and B) after prolonged ciprofloxacin (cipro) treatment. Human peripheral blood T cells from healthy donors were pre-activated by PHA treatment and subsequently cultured for 7 days in presence or absence of 50 μg/ml cipro. Next, T cells were activated via plate-bound anti-CD3 antibody (30 μg/ml) and soluble anti-CD28 antibody (1 μg/ml) for 16 h. Supernatants were harvested and secreted amounts of IL-4 were measured by ELISA.

FIG. 7B shows activation-induced IL-4 secretion in pre-activated peripheral blood T cells of two patients with atopic dermatitis (patient A and B) after prolonged ciprofloxacin (cipro) treatment. Human peripheral blood T cells from patients with atopic dermatitis were pre-activated by PHA treatment and subsequently cultured for 7 days in presence or absence of 50 μg/ml cipro. Next, T cells were activated via plate-bound anti-CD3 antibody (30 μg/ml) and soluble anti-CD28 antibody (1 μg/ml) for 16 h. Supernatants were harvested and secreted amounts of IL-4 were measured by ELISA.

FIG. 8 Freshly isolated peripheral blood T-cells (“day 0”) from 5 healthy donors and 7 patients with atopic dermatitis were pre-activated with PHA and expanded for 7 days in vitro in the presence of 50 μg/ml ciprofloxacin (Cipro). A comparison between T cells (“day 0”) and PHA-pre-activated T cells treated with cipro revealed a significant down-regulation of basal IL-4 levels in healthy controls and patients (P<0.05 (0.03125).

(*) healthy donors and P<0.01 (0.00781)
(**) patients upon treatment.

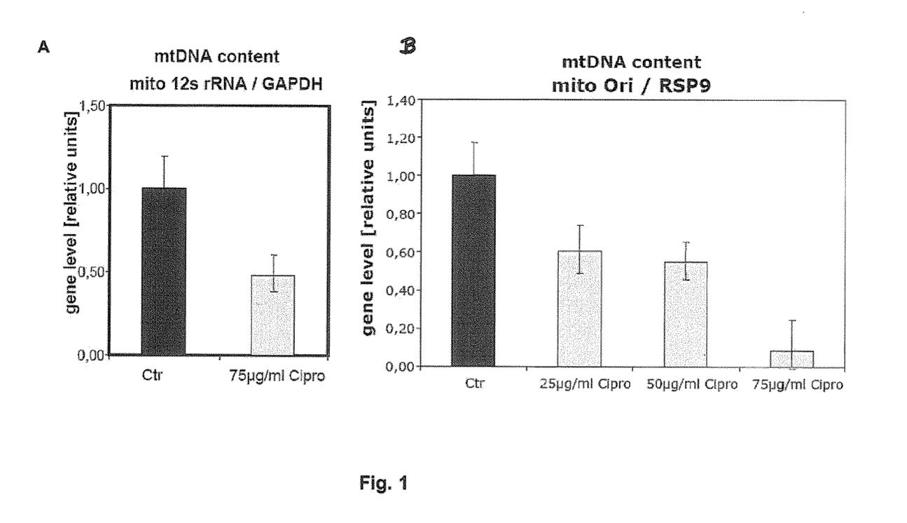

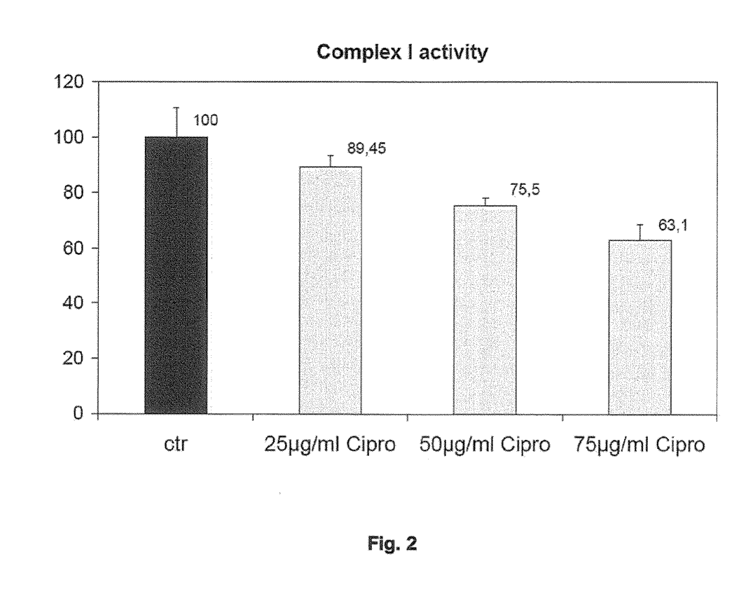

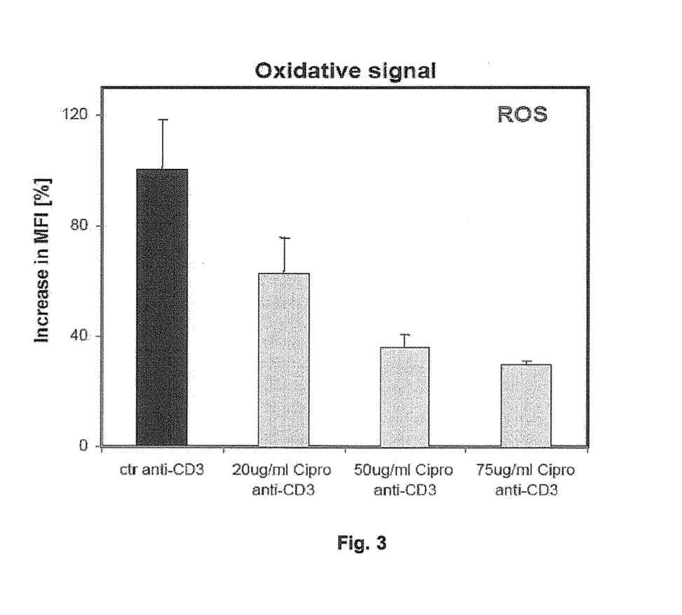

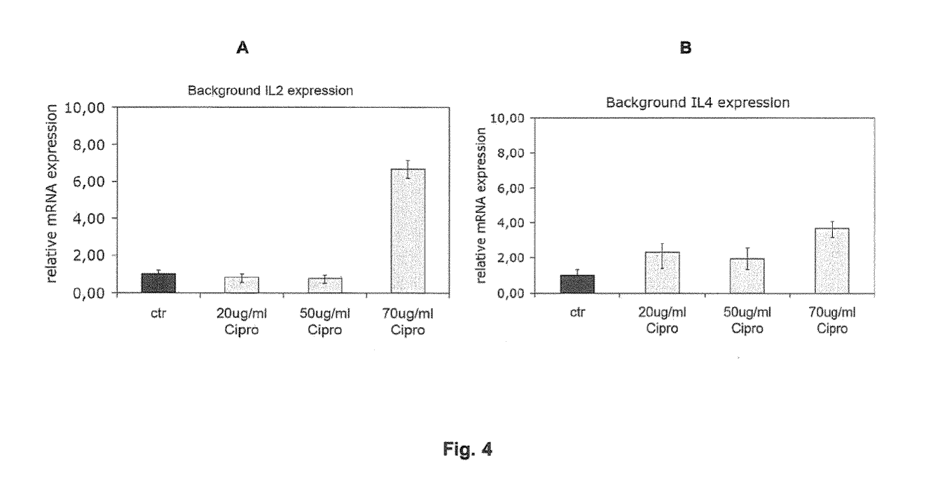

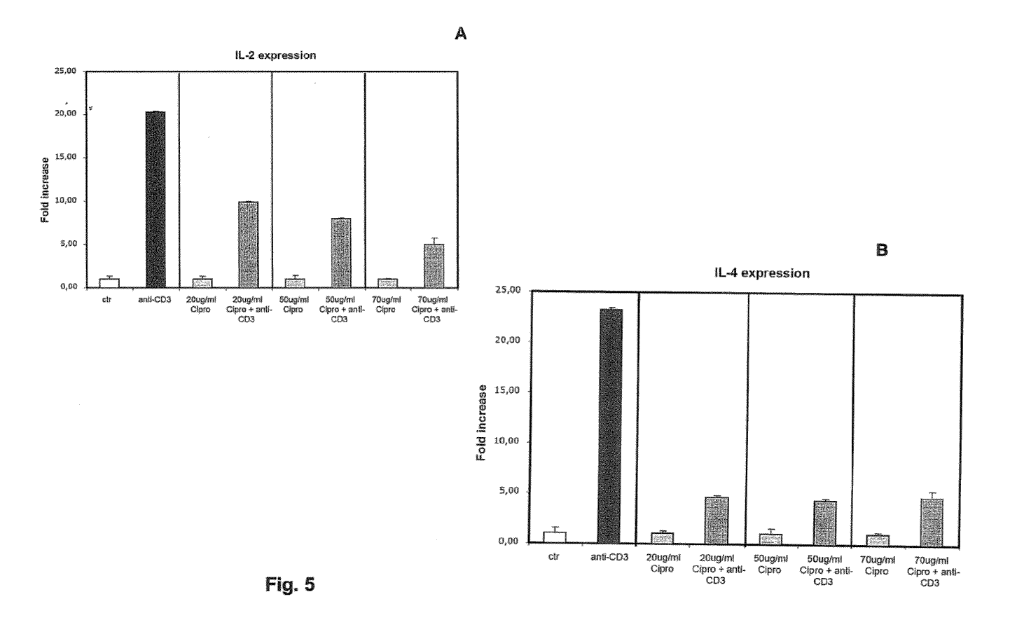

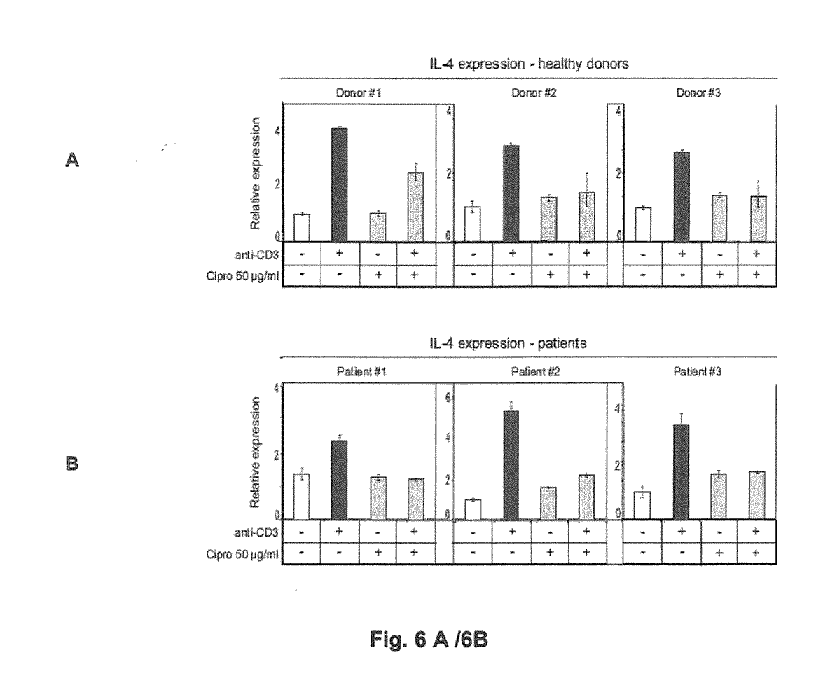

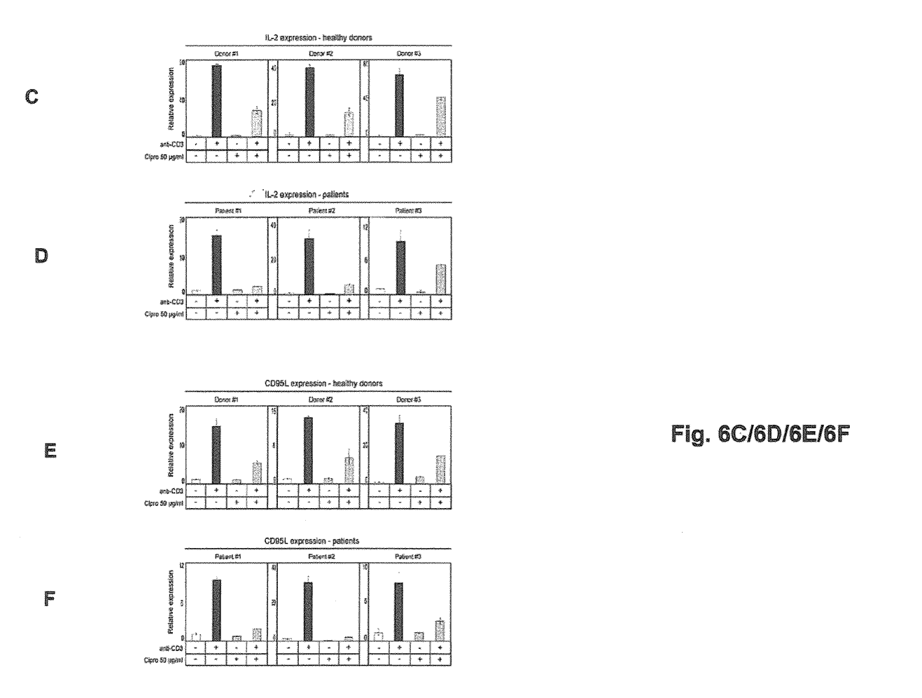

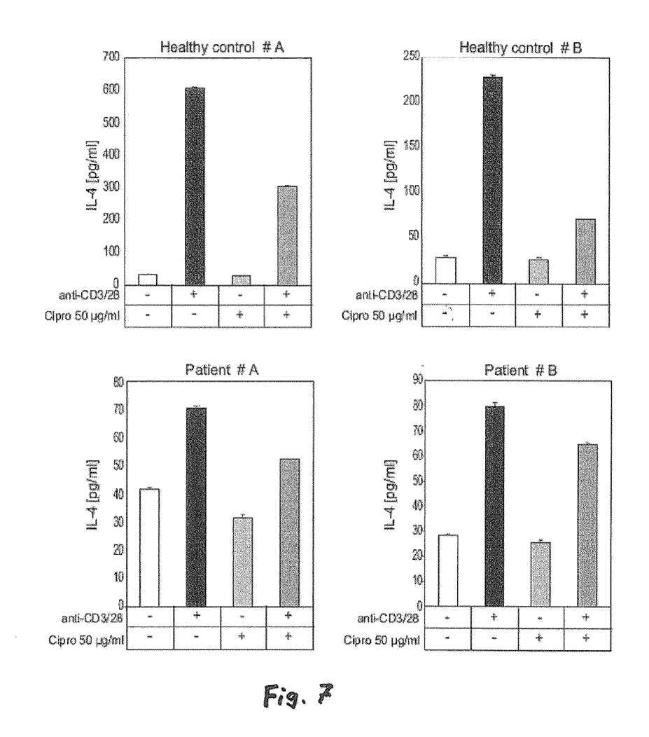

The results presented show clear immunosuppressive effects of prolonged ciprofloxacin treatment on function of pre-activated primary human T-cells. It is further shown that is prolonged ciprofloxacin treatment (7-8 days) leads to mtDNA loss (see FIGS. 1A and B), consequently to a decrease in activity of mitochondrial complex I (see FIG. 2) and the activation-induced oxidative signal (see FIG. 3). Although ciprofloxacin-treatment slightly increases background IL-2 and IL-4 expression (see FIGS. 4A and 4B), the anti-CD 3-induced increase in IL-2 and IL-4 transcripts levels was clearly blocked (see FIGS. 5A and 5B). The same is observed for T-cells isolated from patients with atopic dermatitis. A clear inhibition of TCR induced up-regulation of IL-4, IL-2 and CD95L was detected (see FIGS. 6A to 6F).

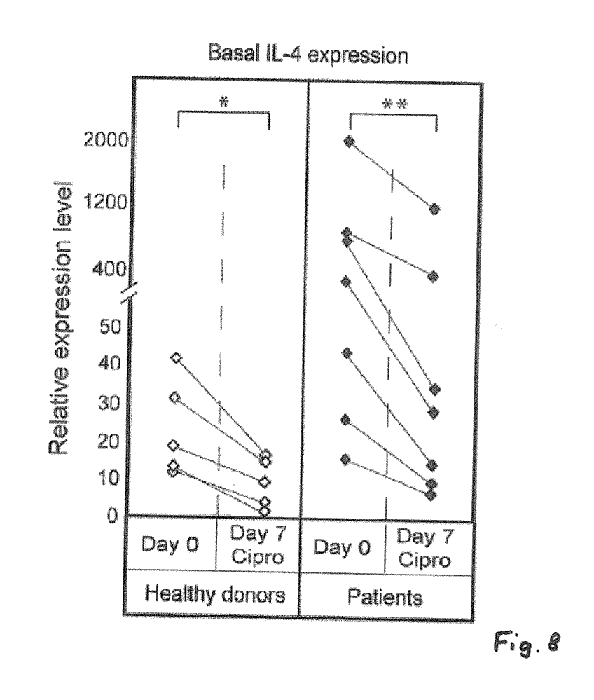

Furthermore, CD3/CD28 triggering-induced IL-4 secretion is also blocked in peripheral blood T-cells of healthy donors and patients with atopic dermatitis. Thus, inhibition of IL-4 transcription is not compensated by translational or post translational cellular mechanisms (sec FIGS. 7A and 7B).

In addition, in T-cells from atopic dermatitis patients a significant down-regulation of IL-4 expression was detected comparing resting T-cells (“day 0”) and PHA-pre-activated T-cells after prolonged ciprofloxacin treatment (FIG. 8). The inhibition of complex I activity by prolonged ciprofloxacin treatment down-regulates basal and activation-induced IL-4 hyper-expression in peripheral blood T-cells of healthy donors and patients with atopic dermatitis, as it is shown in FIG. 6.

Due to the results described above inflammatory diseases like atopic dermatitis, in particular diseases, where pathologic conditions develop from increased IL-4 production by hyperactivated Th2 cells, can effectively treated with prolonged application of fluoroquinolones such as ciprofloxacin.

The present invention is explained in more detail in the following examples.

### EXAMPLE 1

### Materials and Methods

### 1.1 Chemicals

Dichlorodihydrofluorescein diacetate (H2DCF-DA), fluo-4-acetoxymethyl ester (Fluo-4-AM), carboxyfluorescein succinimidyl (CFSE) and BAPTA-AM were obtained from Invitrogen. Ionomycin (Iono) and cyclosporine A (CsA) were purchased from Merck and Ciprofloxacin hydrochloride (Cipro) from Applichem. ALL-acetylcysteine (NAC), phorbol 12-myristatc-13-acetate (PMA), glucose oxidase (GOX), rotenone (Rot) and all other chemicals were supplied by Sigma-Aldrich. FITC-conjugated anti-CD3 antibody was purchased from Becton Dickinson. The monoclonal mouse antibodies (OKT3) against human CD3 and human CD28 (15E8) were prepared.

### 1.2 Patients

T-cells isolated from 9 patients with acute exerbations of long-standing atopic dermatitis were investigated. Blood was drawn before initiation of therapy. As controls, T-cells from normal, age-matched healthy donors were analyzed. Informed consent was obtained from all subjects before inclusion. The study was conducted according to the ethical guidelines of the German Cancer Research Center (DKFZ, Heidelberg) and the Helsinki Declaration.

### 1.3 Isolation of Human Peripheral T-Cells

Human peripheral blood lymphocytes were purified. Homogeneity of the prepared T-cells was verified by staining with FITC conjugated anti-CD3 antibodies followed by fluorescence-activated cell sorting (FACS) analysis and was estimated to be >90%.

1.4 Cell Culture

Jurkat J16-145 cells were derived from the human lymphoblastoid cell line Jurkat J16 8. JurkaT-cells were cultured in IMDM, 10% foetal calf serum (FCS). Freshly isolated resting (“day 0”) or activated (“day 6”) peripheral human T-cells were cultured at a concentration of 2×106 cells/ml in RPMI 1640 (+L-glutaminc), 10% FCS. For activation, “day 0” T-cells were treated with 1 μg/ml phytohemagglutinin (PHA) for 16 h, washed and subsequently cultured in the presence of 25 U/ml IL-2 for 6 (“day 6” T-cells) or 7 days (Ciprofloxacin treatment).

### 1.5 Determination of ROS Generation

Cells were stained with H2DCF-DA (5 μM) for 30 min. Next, cells were then divided and stimulated with either plate-bound anti-CD3 antibody (30 μg/ml) or PMA (10 ng/ml). Treatment was terminated by ice-cold PBS and ROS generation was determined by FACS analysis. If not stated otherwise, ROS generation was quantified as the increase in mean fluorescence intensity (MFI), calculated according to the following formula: increase in

MFI(%)=[(MFI_{stimulated}−MFI_{unstimulated})/MFI_{unstimulated}]×100 9.

### 1.6 Quantitative Real-Time-PCR

Quantitative real-time-PCR was performed using the Power SYBR Green PCR Master Mix (Applied Biosystems). Gene expression was analyzed using the 7500 Real-Time PCR Systems and Sequence Detection Software version 1.2.2 (Applied Biosystems). IL-2 and IL-4 gene expression levels were normalized using glyceraldehyde-3-phosphat dehydrogenase (GAPDH) expression levels as an endogenous reference.

mtDNA content was estimated by gene copy number of the mitochondrial 12S rRNA gene and normalized to the gene copy number of nuclear GAPDH. Induction ratios (X) were calculated using the formula X=2−ΔΔCt, where Ct stands for cycle threshold and ΔCt=Ct gene of interest −Ct reference gene. ΔΔCt is the difference between the ΔCt values of the “induced” samples and the ΔCt of the corresponding “non-induced” sample. The mean induction ratios were calculated. IL-4 basal expression levels in T-cell samples from acute atopic dermatitis patients and healthy donors were compared.

The following primers were used for gene expression analysis:

GAPDH, sense 5′-GCAAATTCCATGGCACCG-3′,
anti-sense 5′-TCGCCCCACTTGATTTTGG-3′;
IL-2, sense 5′-CAACTGGAGCATTTACTGCTG-3′,
anti-sense 5′-TCAGTTCTGTGGCCTTCTTGG-3′;
IL-4, sense 5′-CACAAGCAGCTGATCCGATTC-3′,
anti-sense 5′-TCTGGTTGGCTTCCTTCACAG-3′,
NDUFAFI, sense 5′-GCAGTTTCTGGCACATGG-3′,
anti-sense, 5′-AAAGTAAGTTTCTTCCTGGGCTA-3′.

Primers used for estimation of mtDNA content:

I2S rRNA, sense 5′-GACGTTAGGTCAAGGTGTAG-3′,
anti-sense 5′-CAACTAAGCACTCTACTCTC-3′;
GAPDH, sense 5′-GACCCCTTCATTGACCTCAAC-3′,
anti-sense 5′-CTTCTCCATGGTGGTGAAGA-3′.
CD95L, sense, 5′-AAAGTGGCCCATTTAACAGGC-3′,
antisense, 5′-AAAGCAGGACAATTCCATAGGTG-3′

### 1.7 Determination of IL-2 and IL-4 Secretion

IL-2 and IL-4 concentrations were measured by enzyme-linked immunosorbent (ELISA) assay (BD OptEIA Set Human IL-2/IL-4, Becton Dickinson). Resting peripheral human T-cells (“day 0” T-cells)+/−rotenone (10 μg/ml, 15 min pre-treatment) were stimulated with plate-bound anti-CD3 monoclonal antibody (30 μg/ml) and soluble anti-CD28 monoclonal antibody (1 μg/ml) for 4 h. Next, the supernatants were cleared by centrifugation and the measurements were performed according to the manufacturer's instructions.

### 1.8 Measurement of Enzymatic Activity of Mitochondrial Enzymes

Activities of the respiratory chain single enzyme complexes I and II, as well as of citrate synthase, were measured as described previously with minor modifications. Ciprofloxacin-treated cells were depleted of dead cells via Biocoll (Biochrom) gradient centrifugation. The cell number in different batches of ciprofloxacin-treated cells was equalized. For measurement of the enzymatic activities, samples were prepared as described previously with minor modifications. 4×107 cells were washed with PBS, shock-frozen in liquid nitrogen and thawed on ice. Next, cells were permeabilized by 15 min incubation with 1 ml of 0.015% digitonin (w/v) in respiratory chain (RC) buffer (250 mM sucrose, 50 mM KCl, 5 mM MgCl2, 20 mM Tris-HCl, pH 7.4), washed with RC buffer and centrifuged at 8 000 rpm for 5 min at 4° C. For a single data point the activity measurement was performed three times in triplicates and the average value was calculated. Steady state activity was recorded in a 96-well plate spectrophotometer using a thermostated chamber and a final volume of 300 μl. Enzymatic activities of complex I and complex II were recorded as NADH oxidation at 340-400 nm and as succinate oxidation at 610-750 nm, respectively. Citrate synthase activity was detected after two additional freeze/thaw cycles as 5,5′-dithiobis-(2-nitrobenzoic acid) (DTNB) reduction at 412 nm.

### 1.9 Assessment of Proliferation

After overnight (18 h) incubation with PHA, activated human T-cells were washed, stained with CFSE (1 μM) according to the manufacturer's instructions and treated with different amounts of ciprofloxacin for 7 days. The proliferation was assessed by FACS measurement and calculated as a percentage of the living cells showing reduced CFSE staining (“CFSE low”) due to proliferation-induced dilution of the dye in comparison with non-proliferating cells (“CFSE high”).

### EXAMPLE 2

### Effects of Prolonged Treatment with Ciprofloxacin Human T-Cells

### EXAMPLE 2.1

### Ciprofloxacin-Induced Loss of mtDNA Content

Isolated human peripheral blood T-cells were pre-activated by an overnight treatment with PHA (lectine) and treated with different amounts of ciprofloxacin (Cipro) for 7 days. Next, total cellular DNA was isolated and mtDNA content was estimated using quantitative real-time SYBRGreen PCR. Amount of mtDNA was estimated by gene expression ratio between mitochondrial 12s rRNA gene and nuclear GAPDH (sec FIG. 1A) or a fragment of heavy strand containing the mitochondrial origin of replication (mito Ori) and nuclear gene RSP9 (see FIG. 1B). T-cells from at least three healthy donors were analyzed. The results can be seen in FIGS. 1A and 1B and demonstrate that prolonged ciprofloxacin treatment induce loss of mtDNA content.

### EXAMPLE 2.2

### Impaired Activity of Mitochondrial Respiratory Complex I in Ciprofloxacin-Treated Pre-Activated Human T-Cells

PHA-pre-activated and treated T-cells (see Example 2.1) were shock-frozen in liquid nitrogen. Next, activity of mitochondrial respiratory complex I (NADH-quinone oxidoreductase) was measured by real-time spectrophotometry. After the depletion of dead cells and adjustment to equal cell number in each sample, PHA-pre-activated and ciprofloxacin- (Cipro) treated T-cells (day 7) were shock-frozen in liquid nitrogen. Next, the activity of mitochondrial respiratory complex I was measured by realtime spectrophotometry (15 min, 20° C.) and normalized to total protein content. Presented data were obtained by triplicated measurements of cells from three different donors. The results can be seen in FIG. 2 and demonstrate that loss of mtDNA content leads to impaired activity of mitochondrial respiratory complex I in ciprofloxacin-treated pre-activated human T-cells.

### EXAMPLE 2.3

### Activation-Induced ROS Generation

After PHA-pre-activated and 7 days of Cipro treatment, T-cells were stained with redox sensitive fluorescent probe H2DCF-DA (20 min, 37° C.) and subsequently activated via plate-bound agonistic anti-CD3 antibody (30 μg/ml). The level of activation-induced ROS was assessed by FACS measurement 1 h after activation and calculated as percentage increase in MFI (untreated control set to 100%). T-cells from at least three healthy donors were analyzed. The results can be seen in FIG. 3 and demonstrate that prolonged ciprofloxacin treatment blocks activation-induced ROS generation in preactivated primary human T-cells.

### EXAMPLE 2.4

### Basal IL-2 and IL-4 Expression

Primary human T-cells were PHA-pre-activated and cultured in presence of different amounts of ciprofloxacin for 7 days. Total cellular RNA was isolated and reverse-transcribed. Expression of IL-2 (see FIG. 4A) and IL-4 (see FIG. 4B) was analyzed using quantitative real-time SYBRGreen PCR. IL-2 and IL-4 expression levels were estimated by gene expression ratio between IL-2 or IL-4 gene and GAPDH genes. The results are shown in FIGS. 4A and 4B and demonstrate that prolonged ciprofloxacin treatment increases basal IL-2 and IL-4 expression slightly.

### EXAMPLE 2.5

### Immunosuppressive Effect on Activated Primary Human T-Cells

After prolonged ciprofloxacin treatment (7 days) PHA-pre-activated primary human T-cells were activated with plate-bound anti-CD3 agonistic antibody for 1 h. Total cellular RNA was isolated and reverse-transcribed. Expression of IL-2 (see FIG. 5A) and IL-4 (see FIG. 5B) was analyzed using quantitative real-time SYBRGreen PCR. IL-2 and IL-4 expression levels were estimated by gene expression ratio between IL-2 or IL-4 gene and GAPDH genes. Data are shown as fold increase of induction of gene expression, where anti-CD3 activated cells are compared with respective unstimulated controls. The results to can be seen in FIGS. 5A and 5B and demonstrate that the Ciprofloxacin-induced loss of mtDNA content and inhibition of complex I activity blocks CD3-triggered ROS production and decreases IL-2 and IL-4 expression. Thus prolonged ciprofloxacin treatment has an immunosuppressive effect on activated primary human T-cells.

### EXAMPLE 2.6

### IL-4, IL-2 and CD95L Expression in Peripheral Blood Cells of Healthy Donors or Patients with Atopic Dermatitis

Human peripheral blood T-cells from healthy donors or patients with atopic dermatitis were pre-activated by PHA treatment and subsequently cultured for 7 days in presence or absence of 50 μg/ml ciprofloxacin (Cipro). Next, the cells were activated via plate-bound anti-CD3 antibody for 1 h and the gene expression levels for IL-4 (sec FIGS. 6A and 13), IL-2 (see FIGS. 6C and D) and CD95L (see FIGS. 6E and F) were assayed. The results can be seen in FIG. 6 A to F and demonstrate that the anti-CD3-induced increase in IL-2, IL-4 and CD95L transcripts levels was clearly blocked in T-cells isolated from patients with atopic dermatitis. A clear inhibition of TCR induced up-regulation of IL-4, IL-2 and CD95L was detected.

### EXAMPLE 2.7

### IL-4 Secretion in Peripheral Blood Cells of Healthy Donors or Patients with Atopic Dermatitis

Human peripheral blood T-cells from healthy donors or patients with atopic dermatitis were pre-activated by PHA treatment and subsequently cultured for 7 days in presence or absence of 50 μg/ml cipro. Next, T-cells were activated via plate-bound anti-CD3 antibody (30 μg/ml) and soluble anti-CD28 antibody (1 μg/ml) for 16 h. Supernatants were harvested and secreted amounts of IL-4 were measured by ELISA. The results can be seen in FIGS. 7 A and B and demonstrates that CD3/CD28 triggering-induced IL-4 secretion is blocked in healthy donors and atopic dermatitis patients. Therefore, inhibition of IL-4 transcription is not compensated by translational or post translational mechanisms. Thus, prolonged ciprofloxacin leads to an inhibition of IL-4 protein secretion.

### EXAMPLE 2.8

### Changes of Basal Expression of IL-4

Freshly isolated peripheral blood T cells (“day 0”) from 5 healthy donors and 7 patients with atopic dermatitis were pre-activated with PHA and expanded for 7 days in vitro in the presence of 50 μg/ml ciprofloxacin (Cipro). A comparison between T cells (“day 0”) and to PHA-pre-activated T cells treated with cipro revealed a significant down-regulation of basal IL-4 levels in healthy controls and patients upon treatment (FIG. 8). This result demonstrates that prolonged ciprofloxacin treatment leads to a reduction in basal IL-4 expression (spontaneous hyperexpression) in T-cells from atopic dermatitis patients.

### EXAMPLE 3

1000 g of a cream having the following composition is prepared according to the methods known to the person skilled in the art by mixing of 0.6% of Ciprofloxacin, 0.75% of Span 65® (sorbitans tristearate, detergent, MerckSchuchardtOHG), 2.5% of liquid petrolatum, 1% of Sepigel 305® (Polyacrylamide/C13.14 Isoparaffin/Laureth-7-SEPPIC, thickening and emulsifying agent), 2% of Myrj 52 (Polyoxyethylene 40 stearate, emulsifier, Sigma-Aldrich), 5% of ketostearyl alcohol (50-50), 17% of perhydrosqualene, 3.5% of glycerol, 0.15% of preserving agent, 0.03% of disodium EDTA, 0.5% of triethanolamine, and qs100% of demineralized water. The pH value of the formula is adjusted to 4.5. This cream can be used for skin treatment against dermatitis.

### EXAMPLE 4

500 g of a gel having the following composition is prepared according to the methods known to the person skilled in the art by mixing 0.5% of Ciprofloxacin, 1.5% of Eusolex 232® (Phenylbenzimidazole sulfonic acid, UVB filter, Merck KGaA), 1.2% of Carbomer 934 P® (thickening, suspending and stabilizing substance, Goodrich), 4% of glycerol, 0.2% of triethanolamine, 2% of propylene glycol, 0.6% of xanthan gum, and qs100% of demineralized water. The pH value of the formula is adjusted to 4.6. This gel can be used for skin treatment againstvarious inflammatory diseases.

1.-17. (canceled)

18. A pharmaceutical composition for the treatment and/or prophylaxis of inflammatory diseases or conditions comprising at least one compound of formula (I) 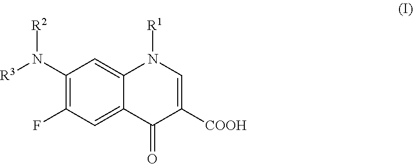
wherein
R¹ denotes hydrogen, or R¹ denotes straight chain C₁-C₆-alkyl, branched C₃-C₆-alkyl, C₃-C₆-cycloalkyl, C₂-C₆-alkenyl, C₂-C₆-alkinyl, all of which may be substituted by one, two or more radicals selected from hydroxyl, C₁-C₆-alkoxy, C₁-C₆-alkylmercapto, and C₁-C₆-alkoxycarbonyl,
R² and R³ are identical or different and denote hydrogen, or R² and R³ are identical or different and denote straight chain C₁-C₆-alkyl, branched C₃-C₆-alkyl, C₃-C₆-cycloalkyl, C₂-C₆-alkenyl, C₂-C₆-alkinyl, all of which may be substituted by one, two or more radicals selected from hydroxyl, C₁-C₆-alkoxy, C₁-C₆-alkylmercapto, and C₁-C₆-alkoxycarbonyl,
or R² and R³ together with the nitrogen atom carrying them form a 3 to 7 membered carboxylic ring which can be interrupted by one or more further hetero-atom selected from N, O, and S, and which may be substituted by one, two or more radicals selected from hydroxyl, C₁-C₆-alkoxy, C₁-C₆-alkylmercapto, and C₁-C₆-alkoxycarbonyl,
or a pharmaceutically acceptable salt, ester or amide or an isomeric or polymorphic form thereof and
at least one pharmaceutically acceptable excipient.

19. A pharmaceutical composition according to claim 18, wherein the composition comprises at least one compound of formula (II) 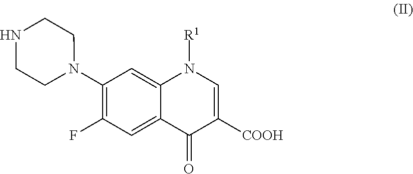
wherein
R¹ denotes hydrogen, or R¹ denotes straight chain C₁-C₆-alkyl, branched C₃-C₆-alkyl, C₃-C₆-cycloalkyl, C₂-C₆-alkenyl, C₂-C₆-alkinyl, all of which may be substituted by a radical selected from hydroxyl, C₁-C₆-alkoxy, C₁-C₆-alkylmercapto, and C₁-C₆-alkoxycarbonyl,
or a pharmaceutically acceptable salt, ester or amide or an isomeric or polymorphic form thereof and
at least one pharmaceutically acceptable excipient.

20. A pharmaceutical composition according to claim 18, wherein the composition comprises 1-cyclopropyl-6-fluoro-1,4-dihydro-4-oxo-7-(1-piperazinyl)-3-quinoline carboxylic acid (ciprofloxacin) or a pharmaceutically acceptable salt, ester or amide or an isomeric or polymorphic form thereof.

21. A pharmaceutical composition according to claim 18, wherein the composition is administered in a prolonged treatment of at least five days.

22. A pharmaceutical composition according to claim 18, wherein the composition is administered in a prolonged treatment of at least five days in an daily dosage in the range of 0.1 to 0.5 mg/kg body weight.

23. A pharmaceutical composition according to claim 18, wherein the pharmaceutical composition is administered in a prolonged topical dermal application of at least 5 days.

24. A pharmaceutical composition according to claim 18, wherein the pharmaceutical composition is administered in a topical dermal application and wherein the composition is selected from emulsion, solution, suspension, lotion, shake lotion, cream, ointment, gel, foam, and transdermal patch and wherein the inflammation diseases or conditions are inflammatory skin diseases or conditions.

25. A pharmaceutical composition according to claim 18, wherein the inflammatory diseases or conditions are allergic inflammation diseases or conditions.

26. A pharmaceutical composition according to claim 18 for use as a medicament, wherein the inflammatory diseases or conditions are T-cell mediated inflammatory diseases or conditions.

27. A pharmaceutical composition according to claim 18 for use as a medicament, wherein the inflammatory diseases or conditions are selected from:
asthma bronchiale, psoriasis, atopic dermatitis, systemic lupus erythematosus (SLE), Sjörgen's syndrome, rheumatoid arthritis, encephalitis and in particular acute disseminated encephalomyelitis (ADEM), Addison's disease, antiphospholipid antibody syndrome (APS), aplastic anemia, autoimmune hepatitis, coeliac disease, inflammatory bowel disease and in particular Crohn's disease, diabetes mellitus (type 1), Goodpasture's syndrome, hyperthyroidism and in particular Graves' disease, Guillain-Barré syndrome (GBS; also called acute inflammatory demyelinating polyneuropathy, acute idiopathic polyradiculoneuritis, acute idiopathic polyneuritis and Landry's ascending paralysis), hypothyroidism and in particular Hashimoto's disease, idiopathic thrombocytopenic purpura, lupus erythematosus, multiple sclerosis, myasthenia gravis, opsoclonus myoclonus syndrome (OMS), optic neuritis, thyroiditis and in particular Ord's thyroiditis, pemphigus, polyarthritis, primary biliary cirrhosis, psoriasis, rheumatoid arthritis, Reiter's syndrome, Sjögren's syndrome, Takayasu's arteritis, temporal arteritis, warm autoimmune hemolytic anemia, vasulitis and in particular Wegener's granulomatosis, alopecia universalis, Behcet's disease, Chagas' disease, chronic fatigue syndrome, dysautonomia including postural orthostatic tachycardia syndrome (POTS), endometriosis, hidradenitis suppurativa, interstitial cystitis, neuromyotonia, sarcoidosis, scleroderma, ulcerative colitis, vitiligo, vulvodynia and graft-versus-host.

28. A pharmaceutical composition according to claim 18, wherein the inflammatory diseases or conditions are inflammatory skin diseases or conditions selected from psoriasis and atopic dermatitis.

29. A method of for the treatment and/or prophylaxis of an inflammatory disease or condition comprising the step of administering to a patient in need thereof an effective amount of a compound of formula (I) 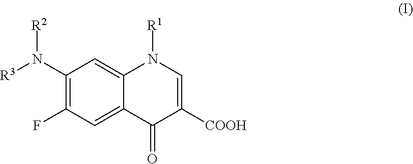
wherein
R¹ denotes hydrogen, or R¹ denotes straight chain C₁-C₆-alkyl, branched C₃-C₆-alkyl, C₃-C₆-cycloalkyl, C₂-C₆-alkenyl, C₂-C₆-alkinyl, all of which may be substituted by one, two or more radicals selected from hydroxyl, C₁-C₆-alkoxy, C₁-C₆-alkylmercapto, and C₁-C₆-alkoxycarbonyl,
R² and R³ are identical or different and denote hydrogen, or R² and R³ are identical or different and denote straight chain C₁-C₆-alkyl, branched C₃-C₆-alkyl, C₃-C₆-cycloalkyl, C₂-C₆-alkenyl, C₂-C₆-alkinyl, all of which may be substituted by one, two or more radicals selected from hydroxyl, alkoxy, alkylmercapto, and alkoxycarbonyl,
or R² and R³ together with the nitrogen atom carrying them form a 3 to 7 membered carboxylic ring which can be interrupted by one or more further hetero-atom selected from N, O, and S, and which may be substituted by one, two or more radicals selected from hydroxyl, alkoxy, alkylmercapto, and alkoxycarbonyl,
or of a pharmaceutically acceptable salt, ester or amide or an isomeric or polymorphic form.

30. A method of claim 29 wherein the inflammatory disease or condition is a T-cell mediated disorder selected from psoriasis, atopic dermatitis and systemic lupus erythematosus (SLE).

31. A dermatological composition comprising at least one compound of formula (I), 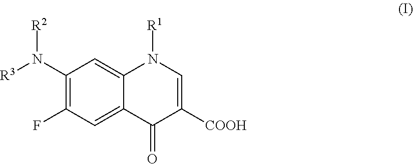
wherein
R¹ denotes hydrogen, or R¹ denotes straight chain C₁-C₆-alkyl, branched C₃-C₆-alkyl, C₃-C₆-cycloalkyl, C₂-C₆-alkenyl, C₂-C₆-alkinyl, all of which may be substituted by one, two or more radicals selected from hydroxyl, C₁-C₆-alkoxy, C₁-C₆-alkylmercapto, and C₁-C₆-alkoxycarbonyl,
R² and R³ are identical or different and denote hydrogen, or R² and R³ are identical or different and denote straight chain C₁-C₆-alkyl, branched C₃-C₆-alkyl, C₃-C₆-cycloalkyl, C₂-C₆-alkenyl, C₂-C₆-alkinyl, all of which may be substituted by one, two or more radicals selected from hydroxyl, alkoxy, alkylmercapto, and alkoxycarbonyl.
or R² and R³ together with the nitrogen atom carrying them form a 3 to 7 membered carboxylic ring which can be interrupted by one or more further hetero-atom selected from N, O, and S, and which may be substituted by one, two or more radicals selected from hydroxyl, alkoxy, alkylmercapto, and alkoxycarbonyl,
or a pharmaceutically acceptable salt, ester or amide or an isomeric or polymorphic form thereof and at least one dermatologically acceptable component.

32. A dermatological composition according to claim 31, wherein the composition comprises from 0.01% to 20% by weight based on the total weight of the composition of at least one compound of formula (I) or a dermatologically acceptable salt thereof.

33. A process for the preparation of a dermatological composition according to claim 31, which comprises the step of mixing a compound of formula (I) or a pharmaceutically acceptable salt, ester or amide or an isomeric or polymorphic form thereof, at least one dermatologically acceptable component and eventually further pharmaceutically active ingredients.

34. A method of for the treatment and/or prophylaxis of an inflammatory skin disease or condition comprising the step of administering to a patient in need thereof an effective amount of a compound of formula (I) 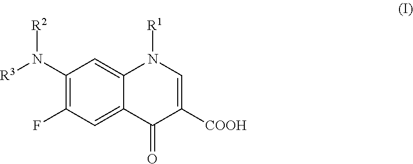
wherein
R¹ denotes hydrogen, straight chain C₁-C₆-alkyl, branched C₃-C₆-alkyl, C₃-C₆-cycloalkyl, C₂-C₆-alkenyl, C₂-C₆-alkinyl, all of which may be substituted by one, two or more radicals selected from hydroxyl, C₁-C₆-alkoxy, C₁-C₆-alkylmercapto, and C₁-C₆-alkoxycarbonyl,
R² and R³ are identical or different and denote hydrogen, straight chain C₁-C₆-alkyl, branched C₃-C₆-alkyl, C₂-C₆-cycloalkyl, C₂-C₆-alkenyl, C₂-C₆-alkinyl, all of which may be substituted by one, two or more radicals selected from hydroxyl, alkoxy, alkylmercapto, and alkoxycarbonyl,
or R² and R³ together with the nitrogen atom carrying them form a 3 to 7 membered carboxylic ring which can be interrupted by one or more further hetero-atom selected from N, O, and S, and which may be substituted by one, two or more radicals selected from hydroxyl, alkoxy, alkylmercapto, and alkoxycarbonyl,
or of a pharmaceutically acceptable salt, ester or amide or an isomeric or polymorphic form thereof.


SEQUENCE LISTING (14 sequences)

SEQ ID NO: 1 - DNA, 18 bp, Homo sapiens
gcaaattccatggcaccg

SEQ ID NO: 2 - DNA, 19 bp, Homo sapiens
tcgccccacttgattttgg

SEQ ID NO: 3 - DNA, 21 bp, Homo sapiens
caactggagcatttactgctg

SEQ ID NO: 4 - DNA, 21 bp, Homo sapiens
tcagttctgtggccttcttgg

SEQ ID NO: 5 - DNA, 21 bp, Homo sapiens
cacaagcagctgatccgattc

SEQ ID NO: 6 - DNA, 21 bp, Homo sapiens
tctggttggcttccttcacag

SEQ ID NO: 7 - DNA, 18 bp, Homo sapiens
gcagtttctggcacatgg

SEQ ID NO: 8 - DNA, 23 bp, Homo sapiens
aaagtaagtttcttcctgggcta

SEQ ID NO: 9 - DNA, 20 bp, Homo sapiens
gacgttaggtcaaggtgtag

SEQ ID NO: 10 - DNA, 20 bp, Homo sapiens
caactaagcactctactctc

SEQ ID NO: 11 - DNA, 21 bp, Homo sapiens
gaccccttcattgacctcaac

SEQ ID NO: 12 - DNA, 20 bp, Homo sapiens
cttctccatggtggtgaaga

SEQ ID NO: 13 - DNA, 21 bp, Homo sapiens
aaagtggcccatttaacaggc

SEQ ID NO: 14 - DNA, 23 bp, Homo sapiens
aaagcaggacaattccataggtg